(19) United States
(12) Patent Application Publication
(10) Pub. No.: US 2009/0177223 A1
(43) Pub. Date: Jul. 9, 2009

(54) System and method for management of type 2 diabetes

(76) Inventor: Tara Chand Singhal, Torrance, CA (US)

Correspondence Address: TARA CHAND SINGHAL, P.O. BOX 5075, TORRANCE, CA, 90510, US

(21) Appl. No.: 12/319,197

(22) Filed: Jan. 2, 2009

Related U.S. Application Data
(60) Provisional application No. 61/009,856, filed on Jan. 3, 2008.

(51) Int. Cl. A61B 17/00

(52) U.S. Cl. 606/202; 606/201

(57) ABSTRACT

For the management of type 2 diabetes, the system has a device that enables applying pressure against the lower abdomen where the pancreas is situated. Applying pressure on the lower abdomen results in pressure being applied to the underlying organ of pancreas, for a limited time in minutes per day. This enables pancreas to function better and thus better able to manage Type 2 diabetes, along with diet and exercise.

### CROSS REFERENCE

This application claims priority on Provisional Application Ser. No. 61/009,856, Titled “A System and Method for Management and Control of Type 2 Diabetes,” Filed On Jan. 3, 2008, By Tara Chand Singhal. The Contents of the Provisional Application Ser. No. 61/009,856 are incorporated herein by reference.

### FIELD OF THE INVENTION

The present invention is directed to a system and method for application of pressure on the lower abdomen, where the pancreas is positioned, for the management of Type 2 diabetes.

### BACKGROUND

Type-2 diabetes is a widely prevalent medical condition. There is no known cure for it. Drugs in combination with diet and exercise are prescribed to treat the condition. Pharmaceutical industry is always researching and marketing new drugs for Type 2 diabetes.

The diabetes is related to the lack of healthy functioning of pancreas as it is the organ that is instrumental in producing insulin for regulating blood sugar. The pancreas is a long tapered gland, which lies across and behind the stomach. Some cells in the pancreas secrete hormones, which regulate the level of glucose in the blood.

The cells of the pancreas are surrounded by many blood vessels into which they secrete hormones (glucagon and insulin) into the blood. Insulin regulates the use of glucose into all the body tissues except the brain. If the pancreas secretes insulin in low quantities, the result is a disease called Type 2 diabetes.

Based on the Indian yoga system of health, it is believed that a certain posture called manduka asana is useful in managing diabetes. The manduka asana requires sitting on the folded legs under the hips, with the two hands forming fists, positioning the fists near the lower abdomen, and bending upper body face down forward towards the knees. This posture applies pressure on the lower abdomen.

In light of the above, it is an objective of the present invention to provide a non-drug based system and method of applying pressure on the lower abdomen that would help in management of Type 2 diabetes.

### SUMMARY

The present invention is directed to a system and method for management of Type 2 diabetes in a human body. The system applies pressure against the lower abdomen, where the pancreas is situated. The system uses a device suitable for placement over the lower abdomen enabling the device to be used to apply pressure against the lower abdomen.

The device may be a substantially flat inflatable container made up of a flexible material such as rubber that holds a quantity of fluid to shape the container to a form a bulge shape that applies pressure on the lower abdomen. The pressure on the lower abdomen may be applied when a person lies face down and positions the device under the lower abdomen.

Alternatively, when the human subject is lying face up or sitting or standing, the device may be positioned near the lower abdomen and strapped around the lower waist with strap means and tightening the strap means may be used to apply pressure on the lower abdomen.

The device-container may have a closeable opening enabling the quantity of the fluid inside the container to be adjusted to grade the different degrees of pressure on the lower abdomen.

The method may involve positioning a device with a elongated bulge shape under the lower abdomen and lying flat on the device for a measured amount of time, enabling the bulge shape to apply pressure on the lower abdomen and thus to the pancreas.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a block diagram that illustrates features of the present invention of system of management of type 2 diabetes;

FIG. 2 is a block diagram that illustrates a version of device of the present invention of system for of management of Type 2 diabetes;

FIG. 3 is a block diagram that illustrates another version of the present invention of system of management of Type 2 diabetes;

FIG. 4 is a flow diagram that illustrates steps of a method of the present invention of system of management of Type 2 diabetes.

### DESCRIPTION

Introduction

The present invention is directed to a system and method for management of Type 2 diabetes in a human body. It is believed, based on the Indian Yoga system, that applying pressure against the lower abdomen and thus to the pancreas inside the lower abdomen improves the functioning of the pancreas.

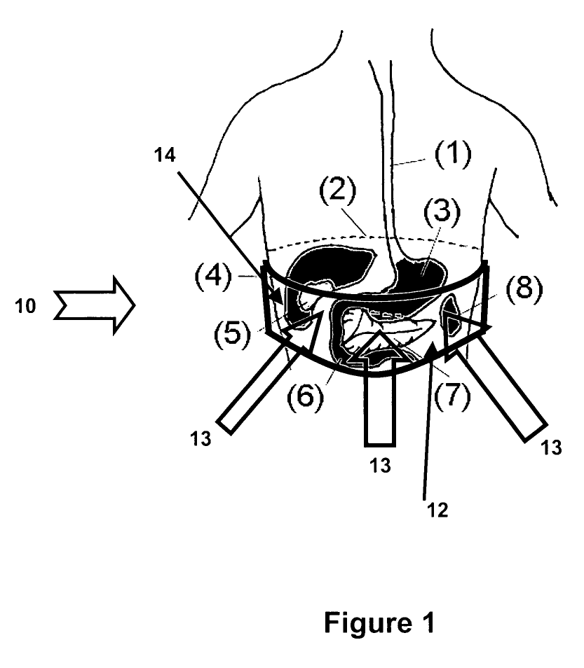

FIG. 1 shows various organs of the body and specifically, the pancreas. FIG. 1 shows esophagus (1), thoracic diaphragm (2), stomach (3), liver (4), gallbladder (5), duodenum (6), pancreas (7) and spleen (8). Pancreas (7) is situated below the stomach (3) and next to the duodenum (6) in lower abdomen 14.

The pancreas 7 is a long tapered gland, which lies across and behind the stomach. The pancreas is a gland organ in the digestive and endocrine systems of vertebrates. It is both exocrine (secreting pancreatic juice containing digestive enzymes) and endocrine (producing several important hormones, including insulin, glucagon, and somatostatin).

With reference to FIG. 1, what is shown is the relative position in human body of pancreas 7. A system 10 has a device 12 that enables applying pressure 13 against the lower abdomen 14, behind which the pancreas 7 is situated.

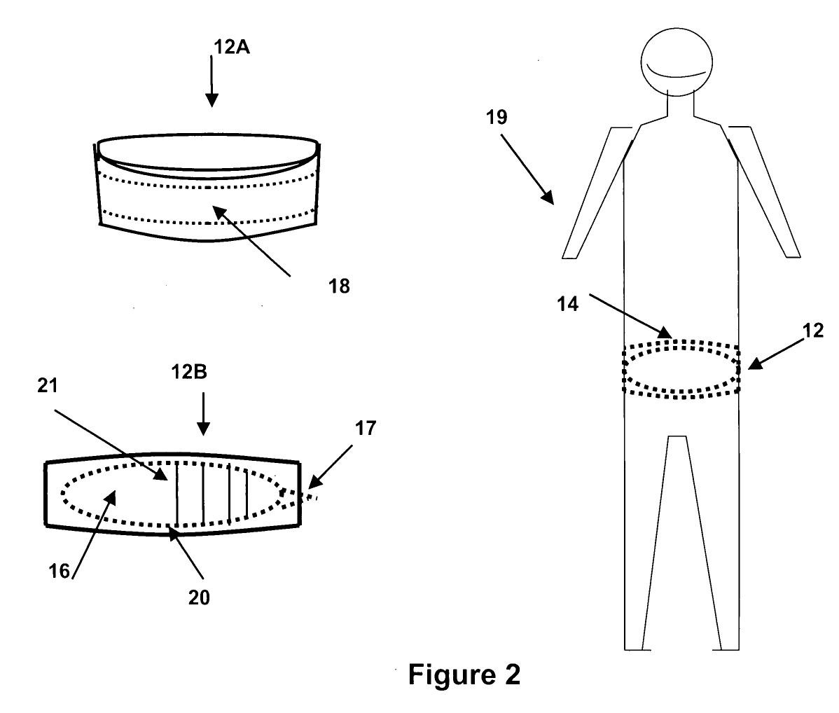

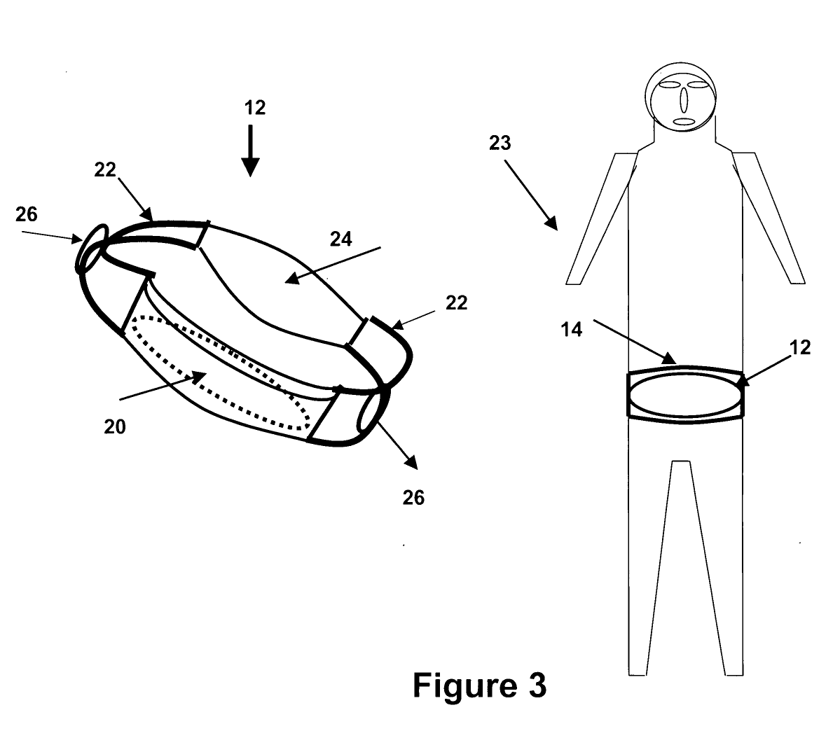

There are different embodiments in how the device 12 of this system 10 may be used for applying pressure on the lower abdomen. One embodiment, as shown in FIG. 2, is lying flat stomach down 19 on the device 12 while placing the device 12 under the lower abdomen 14. Other embodiment as shown in FIG. 3 is in lying on the back face up 23 with device 12 on top of the lower abdomen 14 and using a strap means 22 with the device 12 to apply pressure on the lower abdomen 14. There may be other embodiments that are not ruled out. These embodiments are described herein.

As shown in FIG. 2, the device 12 is shown as a perspective view 12A and a plan view 12B. The device 12 may be an inflatable container 20 made of a flexible material; the container 20 is capable of being filled with a fluid 16 and thus forms a bulge shape 18, that when the human lies face down 19 on top of the device 12 applies pressure to the lower abdomen 14.

The container 20 may have a (i) closeable opening 17 to fill the container 20 with the fluid 16, (ii) has markings 21 to identify the quantity of fluid in the container 20. The quantity of fluid 16 is used to determine the amount of pressure that may be applied to the lower abdomen 14.

A device 12 to manage type 2 diabetes has an inflatable container 20 made of a flexible material which when inflated with a fluid 16, forms an elongated bulge shape 18. The length and the width of the container are sized to cover the lower abdomen 14. When the inflated device-container 12 is placed on the lower abdomen 14 and when a human subject lies face down 19 on the stomach and applies pressure to a pancreas organ in the lower abdomen 14.

The fluid 16 in the inflatable container 20 of device 12 may be water or another suitable fluid that helps create a bulge shape 18 in the inflatable container.

As shown in FIG. 3, the device 12 may have strap means 22 that enable the device 12 to be strapped to the lower abdomen 14 when the human subject is in a position other than lying face down 19 as in FIG. 2, such as, lying flat on the back, face up 23, or standing (not shown), or sitting position (not shown). The straps means 22 may be attached to the two sides of the device and attached to a back strap support means 24 that forms a pressure means on the lower abdomen 14.

The device 12 and the back-surface 24 along with the strap means 22 form a torso ring to be able position the device 12 over the lower abdomen 14, behind which the pancreas 7 is situated. The straps 22 attach to the two sides of the device 12 and are anchored with the back strap surface 24 forming a pressure means on the lower abdomen 14 over the pancreas.

The strap means 22 may have tension adjustment means 26 having a calibration mechanism (not shown) that may include marking on the straps 22 that show the degree of pressure for different size girth near the lower abdomen of various human subjects.

The tension adjustment means may have a calibration mechanism (not shown) that may include marking on the straps that show the degree of compression for different size girth near the lower abdomen. The calibration means may also include a tension device (not shown) in the strap that shows degrees of tension on the strap.

The system 10 of this embodiment includes applying the device 12 by fastening the device on the lower abdomen 14 and lying on the back 23 for a limited time. Alternatively, the system includes applying the device by fastening the device on the lower abdomen and sitting for a limited time.

The container 20 may be placed inside a pouch (not shown) and the position of the pouch is maintained on the lower abdomen by means of straps 22 tied around the back of a human body and to the pouch.

The degree of pressure is calibrated for different degrees of pressure for different body sizes by varying the quantity of the fluid in the container 20. Alternatively, or in addition the degree of pressure may be calibrated for different pressures by adjusting the tension 26 by the straps 22.

In the system of this invention, in this embodiment, as illustrated with FIG. 2, a human subject would position the device under his lower abdomen and lie flat face down on the device for a limited time to be able to apply pressure on the pancreas. Alternatively, as illustrated with FIG. 3, a human subject would place the device 12 on top of his abdomen, tie it around his lower abdomen with the strap means 22 and 24, adjust the pressure with the strap tension means 26 and lie face up.

It is assumed that the bowl movement had been had to be able to better apply compression to the pancreas.

The limited time for applying compression to the pancreas is estimated to be in minutes and could be as an illustration, from one minute to five minutes. The time could be shorter or longer than these and could be determined for individuals by experimentation that has the optimum benefit in improving the functioning of the pancreas. The person would repeat the process every day.

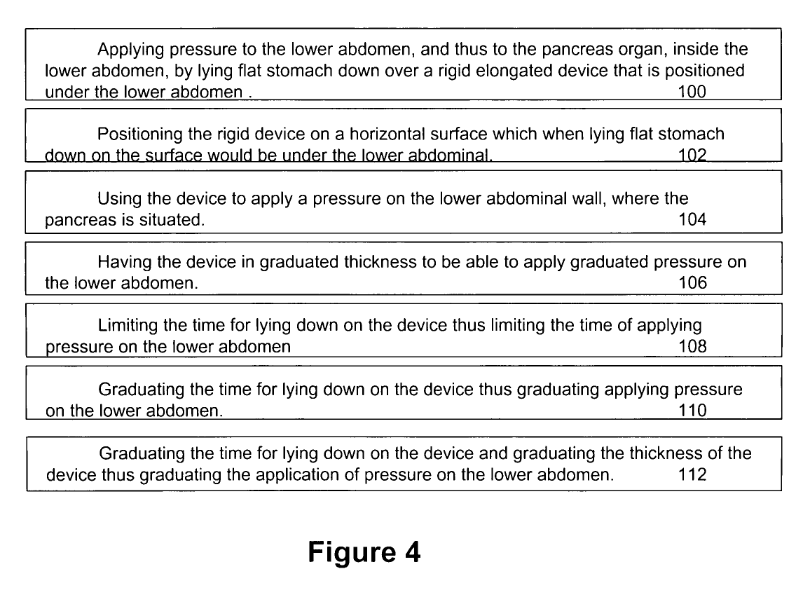

As shown in FIG. 4, a method of managing Type 2 diabetes has the following steps. Not all of these steps may be used or used in the order specified herein.

At step 100, applying pressure to the lower abdomen, and thus to the pancreas organ, inside the lower abdomen, by lying flat stomach down over a rigid elongated device that is positioned under the lower abdomen.

At step 102, positioning the rigid device on a horizontal surface which when lying flat stomach down on the surface would be under the lower abdominal.

At step 104, using the device to apply a pressure on the lower abdominal wall, where the pancreas is situated.

At step 106, having the device in graduated thicknesses to be able to apply graduated pressure on the lower abdomen.

At step 108, limiting the time for lying down on the device thus limiting the time of applying pressure on the lower abdomen.

At step 110, graduating the time for lying down on the device thus graduating applying pressure on the lower abdomen.

At step 112, graduating the time for lying down on the device and graduating the thickness of the device thus graduating the application of pressure on the lower abdomen.

There are two different measurements of blood sugar. One is the fasting glucose blood level, referred to as FBL and the other is an average level over a period of months, referred to as HG A1C. These measurements singly or in combination may be used in measuring the improvement in functioning of the pancreas over time. This system of this invention in conjunction with diet and exercise would help the pancreas function better and reduce the dependency on larger dosages of the diabetes drugs to be able to effectively manage Type 2 diabetes.

In summary, the system and method of this invention may be used for the management of Type 2 diabetes along with diet and exercise and reduce dependence on the use of prescription and over the counter drugs.

While the particular system and method, as illustrated herein and disclosed in detail is fully capable of obtaining the objective and providing the advantages herein before stated, it is to be understood that it is merely illustrative of the presently preferred embodiments of the invention and that no limitations are intended to the details of construction or design herein shown other than as described in the appended claims.

What is claimed is:

1. A system for management of Type 2 diabetes, comprising:
a device that applies a pressure against a lower abdomen and thus to a pancreas organ situated inside the lower abdomen.

2. The system as in claim 1 further comprising:
the device is an inflatable container made of a flexible material, the container is capable of being filled with a fluid and thus forms a bulge shape, that when the human lies face down on top of the device applies pressure to the lower abdomen.

3. The system as in claim 2, further comprising:
the device-container has a (i) closeable opening to fill the container with the fluid, (ii) has markings to identify the quantity of fluid in the container, the quantity of fluid is used to determine the amount of pressure that may be applied to the lower abdomen.

4. The device as in claim 2, further comprising:
the inflatable device-container has length and width that are sized to the size of the lower abdomen.

5. The device as in claim 2, further comprising: the fluid is water.

6. The system as in claim 2, the device further comprising:
the device has strap means that enable the device to be strapped to the lower abdomen when the human subject is in a position other than lying face down, such as, lying flat on the back, or standing, or sitting position.

7. The system as in claim 6, further comprising:
a strap means attached to the two sides of the device and attached to a strap support that forms a pressure means on the lower abdomen.

8. The system as in claim 7, the device further comprising:
the straps means have adjustable tension means to adjust the pressure that may be applied to the lower abdomen.

9. The claim as in 8, further comprising:
the tension adjustment means having a calibration mechanism that includes marking on the straps that show the degree of compression for different size girth near the lower abdomen.

10. A device to mange type 2 diabetes comprising:
an inflatable container made of a flexible material when inflated with a fluid forms an elongated bulge shape, the length and the width of the container are sized to cover the lower abdomen and when placed on the lower abdomen when a human subject lies face down on the stomach and applies pressure to a pancreas organ in the lower abdomen.

11. The device as in claim 10, comprising:
the container is placed inside a pouch and the position of the pouch is maintained on the lower abdomen by means of straps tied around the back of a human body and to the pouch.

12. The device as in claim 11, comprising:
the degree of pressure is calibrated for different degrees of pressure for different body sizes by varying the quantity of the fluid.

13. The device as in claim 11, comprising:
the degree of pressure is calibrated for different pressures by adjusting the tension by the straps.

14. A method of managing type 2 diabetes, comprising the steps of:
applying pressure to the lower abdomen, and thus to the pancreas organ, inside the lower abdomen, by lying flat stomach down over a rigid elongated device that is positioned under the lower abdomen.

15. The method as in claim 14, comprising the steps of:
positioning the rigid device on a horizontal surface which when lying flat stomach down on the surface would be under the lower abdominal.

16. The method as in claim 15, comprising the steps of:
using the device to apply a pressure on the lower abdominal wall, where the pancreas is situated.

17. The method as in claim 16, comprising the steps of:
having the object in graduated thickness to be able to apply graduated pressure on the lower abdomen.

18. The method as in claim 16, comprising the steps of:
limiting the time for lying down on the object thus limiting the time of applying pressure on the lower abdomen.

19. The method as in claim 18, comprising the steps of:
graduating the time for lying down on the device thus graduating applying pressure on the lower abdomen.

20. The method as in claim 19, comprising the steps of:
graduating the time for lying down on the device and graduating the thickness of the device thus graduating the application of pressure on the lower abdomen.
